(19) United States
(12) Patent Application Publication
(10) Pub. No.: US 2009/0306562 A1
(43) Pub. Date: Dec. 10, 2009

(54) TRAINING APPARATUS AND METHODS

(76) Inventor: Keith Welch, Forest Lake, MN (US)

Correspondence Address: MUETING, RAASCH & GEBHARDT, P.A., P.O. BOX 581336, MINNEAPOLIS, MN, 55458-1336, US

(21) Appl. No.: 12/133,956

(22) Filed: Jun. 5, 2008

(51) Int. Cl. A61F 5/00

(52) U.S. Cl. 602/17

(57) ABSTRACT

Apparatus and methods to signal to a user that his/her head is not upright and methods for using such apparatus. For example, the present invention may be an apparatus that includes a tapered resilient block locatable underneath the mandible of a user and retention apparatus to maintain the position of the tapered resilient block.

### BACKGROUND

The present invention relates generally to at least training apparatus and methods. More particularly, the present invention pertains to at least apparatus and methods that signal to a user that his/her head is not upright.

Due to the nature of fast-paced sports such as, e.g., hockey, injuries may occur when a player fails to maintain his/her head upright. For example, during the course of a hockey game, players who skate with their heads looking downward may have a higher risk of collision. Additionally, a hockey player who does not need to look down to control the puck has an advantage over an opponent who needs to look down.

Although these examples are described with reference to hockey, it is desirable in many other sports and activities for a user to keep his/her head upright. For example, a dancer may want to keep perfect form while performing, a basketball or soccer player may want to be able to control the ball without looking downward, etc. Further, for example, babies often need help to support their head.

Apparatus has been developed to assist a user in maintaining his/her head upright. For example, U.S. Pat. No. 5,135,446, issued 4 Aug. 1992 to Cooley and entitled “Training Aid for Skating,” describes a training aid for skaters that includes an enlarged block made from a soft resilient material and including an elongated exterior wall having an upwardly facing exterior surface. The enlarged block is used for obstructing the skater's vision of his skates and a portion of the ice immediately forwardly thereof.

However, such apparatus as described in U.S. Pat. No. 5,135,446 has disadvantages. For example, the apparatus is designed to extend beyond the chin of the user (preferably 8 to 10 inches) to block the user's downward vision. Such extension may be undesirable when it becomes necessary to look downward, e.g., to know where and how a person is falling.

### SUMMARY

In one or more embodiments, the present invention may be an apparatus to signal to a user that his/her head is not upright.

In one embodiment of a training apparatus according to the present invention, the training apparatus includes a resilient tapered block positionable underneath the mandible of a user, wherein the resilient tapered block is compressible under application of a force from a normal state to a compressed state, and wherein the resilient tapered block returns to the normal state when the force is removed. The resilient tapered block may include a proximal surface, wherein the proximal surface is positioned adjacent the neck of the user when the resilient tapered block is positioned underneath the mandible of the user. The tapered block may also include a distal surface and upper and lower divergent surfaces, wherein the upper and lower divergent surfaces diverge such that the distance therebetween increases when moving from the proximal surface to the distal surface, wherein the upper divergent surface is adjacent the mandible of the user when the resilient tapered block is positioned underneath the mandible of the user with the proximal surface adjacent the neck of the user, wherein the upper divergent surface and the distal surface intersect along a mandibular junction of the resilient tapered block, and further wherein the mandibular junction is located under the mandible of the user when the resilient tapered block is positioned underneath the mandible of the user. The training apparatus further includes retention apparatus configured to position the resilient tapered block underneath the mandible of the user, wherein the retention apparatus is configured to be coupled about at least a portion of the neck of the user.

In another embodiment of a training apparatus according to the present invention, the training apparatus is couplable to a neck guard apparatus and may include a resilient tapered block like that described above. However, the tapered block further may include a fastener apparatus located on the proximal surface configured to couple the resilient tapered block to the neck guard.

One embodiment of a method according to the present invention provides for using a training apparatus. The training apparatus may include a resilient tapered block (such as that described above) positionable underneath the mandible of a user and retention apparatus configured to position the resilient tapered block underneath the mandible of the user. The method further includes locating the resilient tapered block underneath the mandible of the user, wherein the proximal surface is positioned adjacent the neck of the user, wherein the upper divergent surface is adjacent the mandible of the user, and further wherein the mandibular junction is located under the mandible of the user. Still further, the method includes coupling the retention apparatus about at least a portion of the neck of the user.

In another embodiment according to the present invention, a support apparatus is provided. The support apparatus includes a resilient shaped block positionable underneath the mandible of a user, wherein the resilient shaped block is compressible under application of a force from a normal state to a compressed state, and further wherein the resilient shaped block returns to the normal state when the force is removed. The resilient shaped block includes a proximal surface extending along a length of the resilient shaped block, wherein the proximal surface is positioned adjacent the neck of the user when the resilient shaped block is positioned underneath the mandible of the user. The shaped block also may include a distal surface and upper and lower surfaces extending from the proximal surface to the distal surface, wherein the upper surface is concave shaped, wherein the upper surface is adjacent the mandible of the user when the resilient shaped block is positioned underneath the mandible of the user, wherein the upper surface and the distal surface intersect along a mandibular junction of the resilient shaped block, and further wherein at least a portion of the mandibular junction is located under the mandible of the user when the resilient shaped block is positioned underneath the mandible of the user. The support apparatus further includes retention apparatus configured to position the resilient shaped block underneath the mandible of the user, wherein the retention apparatus is configured to be coupled about at least a portion of the neck of the user.

The above summary of the present invention is not intended to describe each embodiment or every implementation of the present invention. Advantages, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a perspective view of an exemplary embodiment of a training apparatus worn by a user according to the present invention.

FIG. 2 is a front view of the exemplary embodiment of the training apparatus as shown in FIG. 1 according to the present invention.

FIG. 3 is a top view of the exemplary embodiment of the training apparatus as shown in FIG. 1 according to the present invention.

FIG. 4 is a side view of the exemplary embodiment of the training apparatus as shown in FIG. 1 according to the present invention.

FIG. 5 is a perspective view of another exemplary embodiment of a training apparatus worn by a user according to the present invention where a resilient tapered block is detachable or removable from a neck guard.

FIG. 6 is an exploded perspective view of the exemplary embodiment of the training apparatus as shown in FIG. 5 according to the present invention.

FIG. 7 is a front view of the exemplary embodiment of the detachable resilient tapered block as shown in FIG. 5 according to the present invention.

FIG. 8 is a top view of the exemplary embodiment of the detachable resilient tapered block as shown in FIG. 5 according to the present invention.

FIG. 9 is a side view of the exemplary embodiment of the detachable resilient tapered block as shown in FIG. 5 according to the present invention.

FIG. 10 is a perspective view of another exemplary embodiment of an apparatus worn by a user under the mandible according to the present invention.

FIG. 11 is another perspective view of the exemplary embodiment of the apparatus as shown in FIG. 10 according to the present invention.

FIG. 12 is a front view of the exemplary embodiment of the apparatus as shown in FIG. 10 according to the present invention.

FIG. 13 is a side view of the exemplary embodiment of the apparatus as shown in FIG. 10 according to the present invention.

FIG. 14 is a top view of the exemplary embodiment of the apparatus as shown in FIG. 10 according to the present invention.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention. Unless stated otherwise herein, the figures of the drawing are rendered primarily for clarity and thus may not be drawn to scale.

As used herein, “a,” “an,” “the,” “at least one,” and “one or more” are used interchangeably The term “and/or” (if used) refers to one or all of the listed elements or a combination of any two or more of the listed elements.

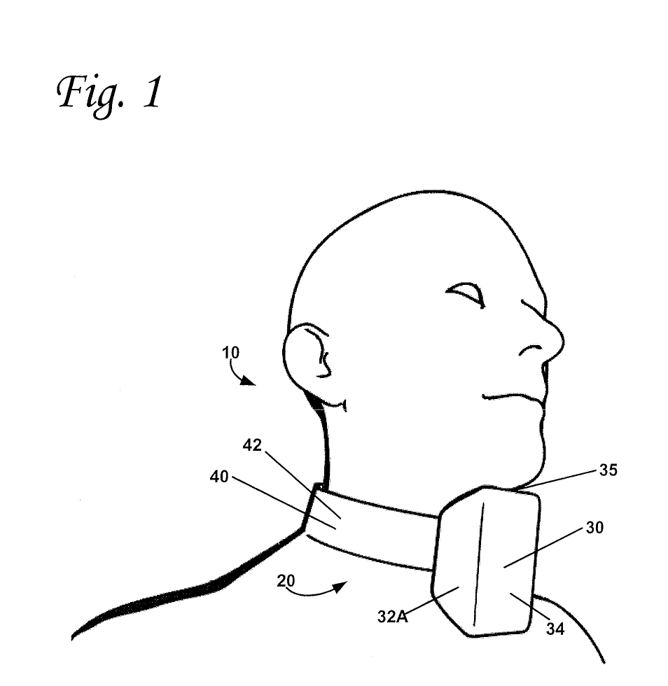

An exemplary embodiment of a training apparatus 20 worn by a user 10 according to the present invention is shown in FIG. 1. The training apparatus 20 may include a resilient tapered block 30 and a retention apparatus 40.

The training apparatus 20 may be worn by a user to signal to the user that his/her head is not upright. For example, the resilient tapered block 30 may be positioned underneath the mandible of a user such that when the user bends his/her neck downwardly from an upright position, the user's mandible will contact the resilient tapered block 30. As such, whenever a user bends his/her neck downwardly, the user's mandible will contact the block. This contact may signal to the user that he/she should move his/her head back to an upright position (i.e., a “heads-up” position).

Further, the resilient tapered block 30 may be formed of resilient material such that the resilient tapered block is compressible under application of a force from a normal state to a compressed state, and the resilient tapered block returns to the normal state when the force is removed. A “normal state” may be defined as the shape and volume of the resilient block before any external forces are applied to it (except, e.g., natural forces include gravity, air pressure, etc.). In one or more embodiments, the resilient tapered block 30 may be compressible to a compressed state that occupies about 90% or less volume, 80% or less volume, 50% or less volume, and/or 30% or less volume than occupied by the resilient tapered block 30 in the normal state.

For example, the resilient tapered block 30 may be compressed by the mandible of the user if the user does not keep their head in an upright position (e.g., a force applied by or through the mandible). If the user continues to bend his/her neck downwardly, the user's mandible may compress the resilient block. Such compression may further signal to the user that he/she should resume an upright, “heads-up” position. In at least one embodiment, the compression of the resilient tapered block may push back against the mandible of the user to force the head of the user into an upright position. Further, as described previously herein, after the user moves his head back to an upright position, the resilient tapered block will return to a normal state.

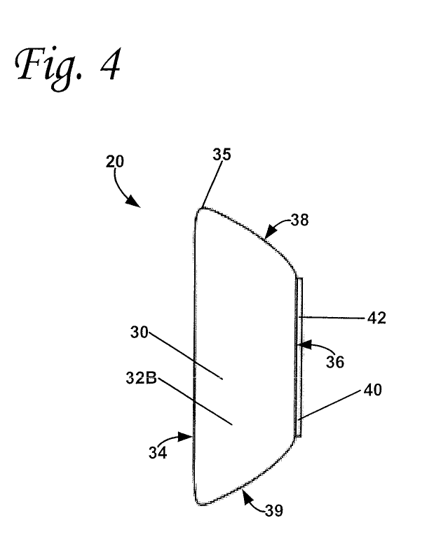

The resilient tapered block 30 may be tapered, e.g., the block 30 may include at least two surfaces (e.g., surfaces 38, 39) extending from a proximal surface 36 to a distal surface 34 that diverge from each other as best shown in FIG. 4. The side view of the resilient tapered block 30 of FIG. 4 shows upper surface 38 and lower surface 39 extending from the proximal surface 36 to distal surface 34. At least portions of the upper and lower surfaces 38, 39 diverge such that the distance between such surfaces increases when moving from the proximal surface 36 to the distal surface 34. In other words, the upper and lower surfaces 38, 39 diverge to taper the block 30. When the resilient tapered block 30 is positioned underneath the mandible of the user, the upper surface 38 may be adjacent the mandible of the user 10. Although the embodiment depicted in FIGS. 1-4 only shows two diverging surfaces, namely upper surface 38 and lower surface 39, the present invention may provide the taper using more than two surfaces.

Further, the upper divergent surface 38 and the distal surface 34 intersect along a mandibular junction 35 of the resilient tapered block 30 (shown in FIGS. 1-4). The resilient tapered block 30 may be sized such that when the resilient tapered block 30 is positioned underneath the mandible of the user 10 (e.g., does not extend forward of the chin of the user), the mandibular junction 35 is located under the mandible of the user 10 (e.g., does not extend forward of the chin of the user). Further, the resilient tapered block 30 may be sized such that the downward vision of the user 10 is not impaired, which may occur if the block extended forward of the chin of the user. Still further, the resilient tapered block 30 may be sized such that when a user bends his/her neck downwardly, the resilient tapered block 30 compresses instead of moving away from the neck of the user 10, which may occur if the block extends past the chin of the user or is very wide.

In one embodiment, the proximal surface 36 extends between a first side surface 32A and a second side surface 32B along axis 37 of the tapered block 30. When the resilient tapered block 30 is positioned underneath the mandible of the user 10, the proximal surface 36 is positioned adjacent the neck of the user 10. In the embodiment depicted in FIGS. 1-4, the distal surface 34 has a larger area than the proximal surface 36 due in part, at least in one embodiment, to the diverging upper and lower surfaces 38, 39 tapering the block 30.

The distal surface 34 may be a surface opposite and substantially parallel the proximal surface 36. In one or more embodiments, the depth 39 between the proximal surface 36 and the distal surface 34 measured orthogonally from the proximal surface 36 may be less than about 3 inches, and even less than about 2 inches. Further, the depth is generally greater than 1 inch. Further, in at least one embodiment, the distal surface 34 and the proximal surface 36 may not be parallel. In these “non-parallel” embodiments, the depth of the taper block 30 may be measured orthogonally from the proximal surface 36 to a plane parallel thereto containing a point of the distal surface 34 farthest from the proximal surface 36.

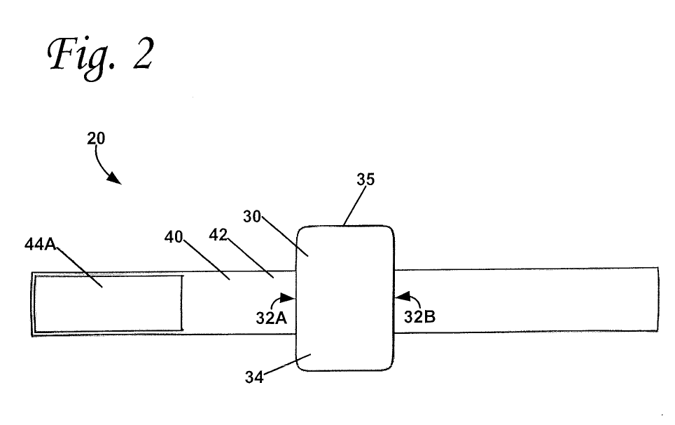

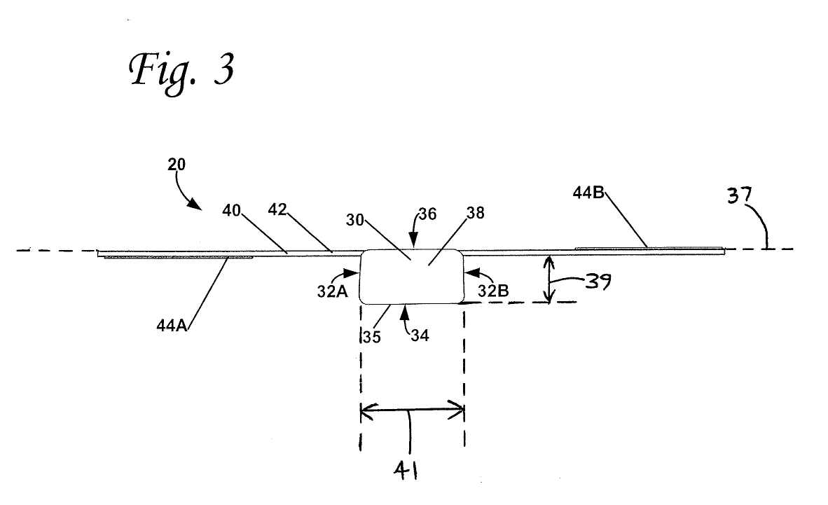

As shown in FIGS. 2-3, the resilient tapered block 30 further includes the first side surface 32A and the second side surface 32B. The first side surface 32A and the second side surface 32B may be substantially parallel. In one or more embodiments, the width 41 of the tapered block 30 between the first side surface 32A and the second side surface 32B measured in a direction parallel to axis 37 is less than about 12 inches, less than about 6 inches, less than about 4 inches or less than about 3 inches. Further, for example, in one or more embodiments, the width is generally more than about 1 inch, and may be more than about 2 inches. Further, in at least one embodiment, the first and second side surfaces 32A, 32B may not be parallel. In these “non-parallel” embodiments, the width 41 is measured in a direction parallel to axis 37 between a first and second plane (both orthogonal to axis 37) that contain points, respectively, of the first and second side surfaces 32A, 32B that are farthest from each other.

The first side surface 32A, the second side surface 32B, the proximal surface 34, the distal surface 36, the upper surface 38, and the lower surface 39 as shown are each substantially planar. In other embodiments, however, each of the first side surface 32A, the second side surface 32B, the proximal surface 34, the distal surface 36, the upper surface 38, and the lower surface 39 may be rounded, jagged, wavy, etc. and/or may include multiple different surfaces, textures, undulations, etc. For example, the diverging upper surface 38 and lower surface 39 may each have a rounded surface. Also, although the side surfaces 32A & 32B as shown are substantially parallel to each other, at least in one embodiment, the side surfaces need not be parallel to each other (e.g., they may also diverge with the distance increasing between them from the proximal surface to the distal surface). Further, for example, the side surfaces may be provided as part of the distal surface and not be separately present (e.g., a round distal surface extending from one end of the proximal surface to the other but the block still tapered by the upper and lower diverging surfaces).

The training apparatus 20 may include retention apparatus 40 configured to position the resilient tapered block 30 underneath the mandible of the user 10 and configured to be coupled about at least a portion of the neck of the user 10. As shown in FIGS. 1-4, the retention apparatus 40 includes one or more band elements 42 and hook and loop closures 44A & 44B. The band elements 42 may be operable to maintain the position of the resilient tapered block 30 underneath the mandible of a user during athletic activity, repetitive compressions/decompressions, etc. The one or more band elements, for example, may be a single band with the tapered block 30 attached thereto or, for example, may include two band elements extending from sides of the tapered block 30. Various configurations of such band elements may be suitable for coupling about at least a portion of the neck. The one or more band elements 42, as well as the tapered block 30, may be formed of a skin-compatible material such as, e.g., cotton, wool, nylon, polymer, neoprene, etc. Further, the one or more band elements 42 may be resilient, i.e., the band 42 may be capable of returning to original form or position after being bent, compressed, stretched, etc. In at least one embodiment, the one or more band elements 42 may not wrap completely around a user's neck and, instead, may wrap around about three quarters of the circumference of the neck of a user but be resilient such that the band retains its position around the user's neck (e.g., use of a covered thin memory metal material). Further, in at least another embodiment, the band 42 may be an elastic band, a neck guard, or any other suitable apparatus that may hold the block 30 in place. Yet further, for example, the block 30 may be held in place by one or ore straps or band elements attached or removably attached to the helmet and/or cage associated therewith (e.g., such as with use of hook and loop closures, snaps, etc.).

The retention apparatus 40 may include hook and loop closures 44A & 44B (e.g., Velcro) that may be attached to each other. For example, a user may couple the training apparatus to his/her neck by grasping each end of the band 42 towards each hook and loop closure, extending each end around his/her neck, and pressing the hook and loop closures 44A & 44B together. The hook and loop closures 44A & 44B may form a connection such that the training apparatus 20 will not fall off during athletic activity. In one embodiment, the training apparatus 20 may be removed by grasping the resilient tapered block 30 and pulling forwards. Further, for example, the hook and loop closures may be on a first band element (e.g., on the same side of the element) extending from one side of the block with a second band element on the other side having a loop attached thereto. As such, the first band element may be inserted into the loop and pulled tight around the user's neck prior to bringing the hook and loop closures together (e.g., as such, no hook and loop closures are adjacent the user's neck). Other embodiments of the present invention may have retention apparatus that includes different closure mechanisms such as, e.g., buckles, snaps, clasps, laces, etc.

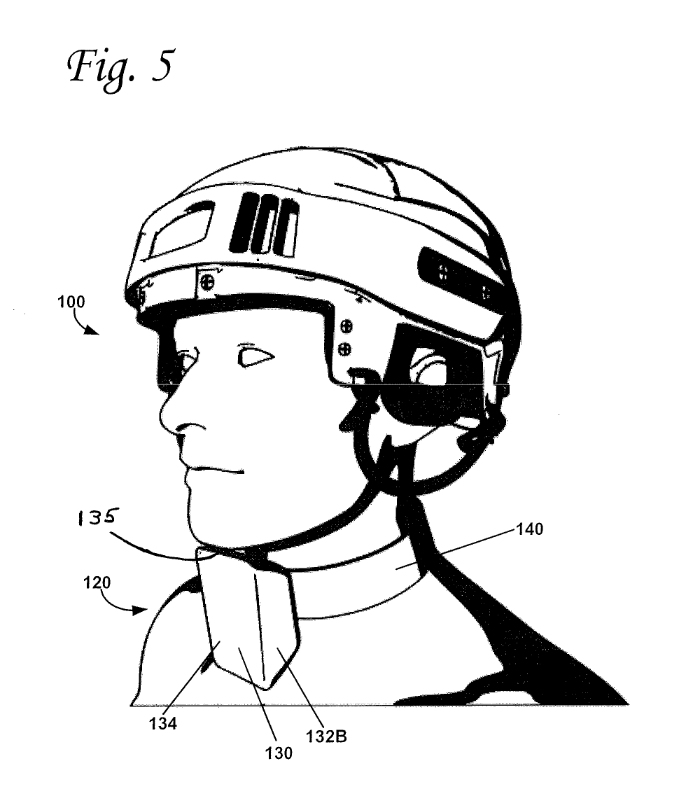

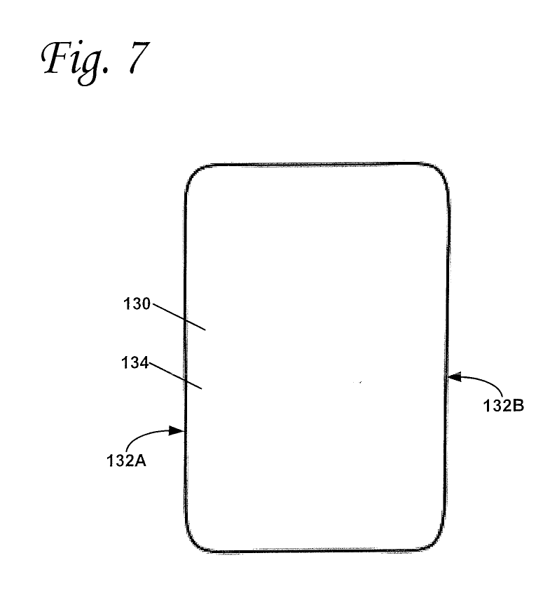

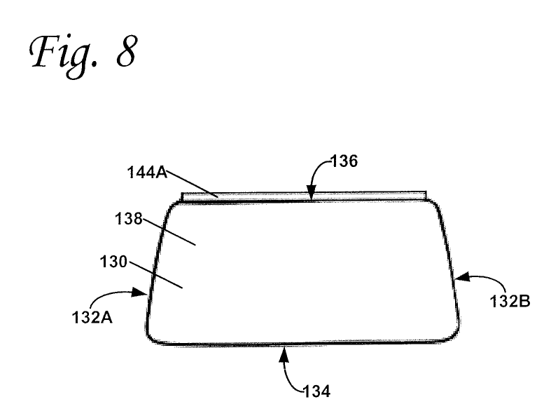

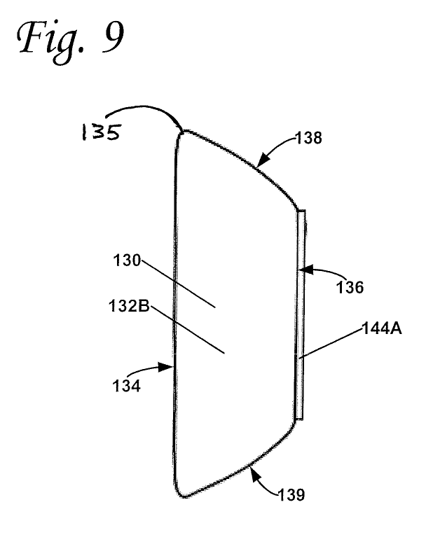

Another embodiment of a training apparatus according to the present invention is a training apparatus 120 worn by a user 100 as depicted in FIGS. 5-9. Training apparatus 120 may be couplable to a neck guard 140. Generally, neck guards are protective bands that extend around a user's neck for protecting the user's neck from harm and are often required use for participation in hockey leagues. Use or adaptation of any neck guard is contemplated according to the present invention (e.g., any conventional neck guard adapted with mounting apparatus, any configuration of neck guard, etc.).

The training apparatus 120 may include a detachable resilient tapered block 130. The detachable resilient tapered block 130 may be similar to the resilient tapered block 30 as described herein with reference to FIGS. 1-4. For example, the detachable resilient tapered block 130 includes first and second side surfaces 132A & 132B, distal and proximal surfaces, 134 & 136, and upper and lower surfaces 138 & 139 that may be substantially similar to the first and second side surfaces 32A & 32B, distal and proximal side surfaces, 34 & 36, and upper and lower surfaces 38 & 39 of resilient tapered block 30. As such, for simplicity, further description on the details of block 130 shall not be provided.

Further, like described with reference to FIGS. 1-4, at least in one embodiment, the upper divergent surface 138 and the distal surface 134 intersect along a mandibular junction 135 of the resilient tapered block 130. The resilient tapered block 130 may be sized such that when the resilient tapered block 130 is positioned underneath the mandible of the user 100 (e.g., attached to a neck guard), the mandibular junction 135 is located under the mandible of the user 100 (e.g., does not extend forward of the chin of the user).

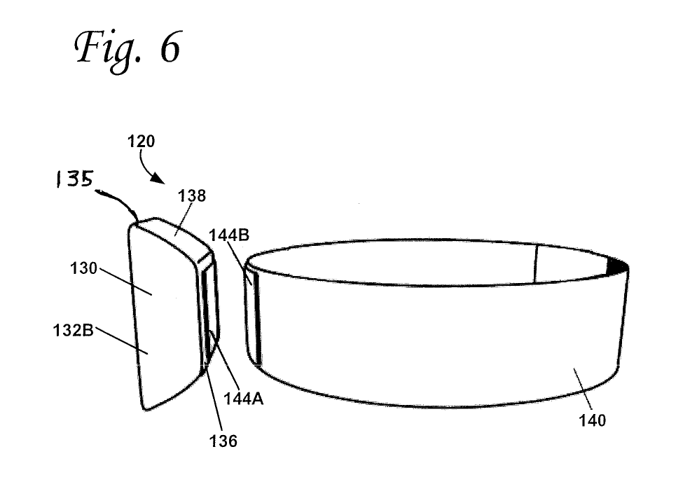

The detachable resilient tapered block 130, however, is designed to be, removably mountable on the neck guard 140 as shown in FIG. 6. The detachable resilient tapered block 130 may include fastener apparatus 144A located on the proximal surface 136 configured to be couplable to corresponding fastener apparatus 144B located on the neck guard 140. The fastener apparatus 144A & 144B may be hook and loop closures (e.g., Velcro) or any other structure for attaching the block 130 to the neck guard 140. For example, such fastener apparatus may allow a user to easily and quickly attach and detach the block 130, yet maintain position of the detachable resilient tapered block 130 underneath the mandible of a user during athletic activity, repetitive compressions/decompressions, etc. For example, the fastener apparatus 144A may be a sleeve through which the neck guard may be inserted (as such, a fastener apparatus on the neck guard may not be required), may be elements foldable over the neck guard 140 suitable to hold the tapered block 130 in place, or may include any other suitable fastener technique.

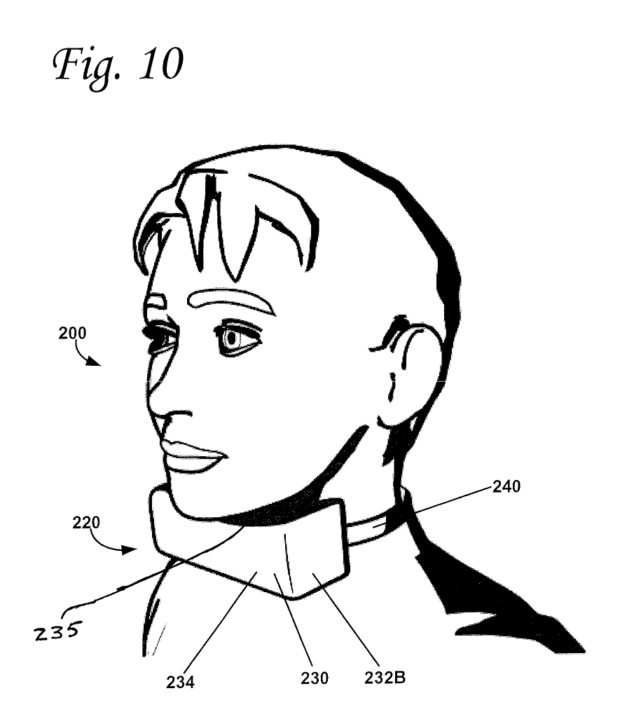

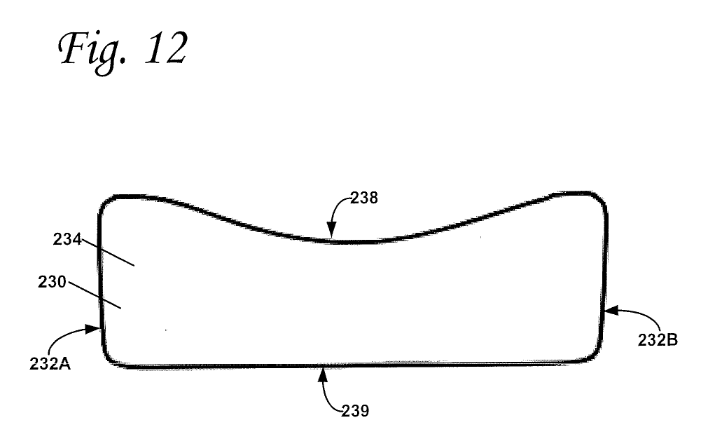

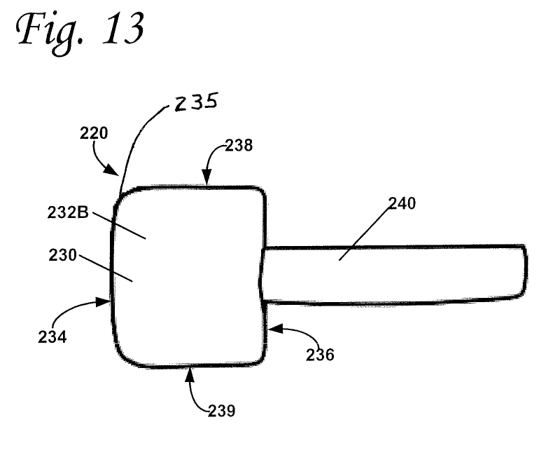

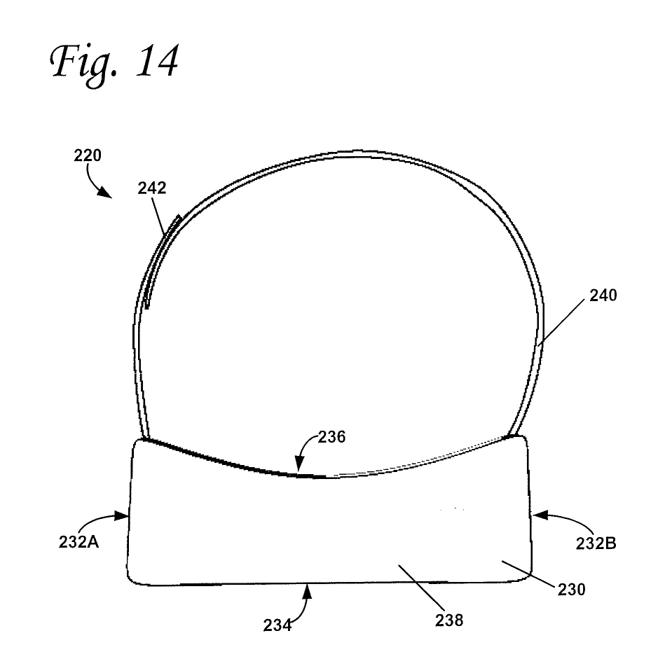

An exemplary embodiment of a support apparatus 220 worn by a user 200 according to the present invention is shown in FIGS. 10-14. The support apparatus 220 may include a resilient shaped block 230 and retention apparatus 240. The support apparatus 220 may be designed to assist a user to maintain his/her head upright and/or to support the user's head. In at least one embodiment, the support apparatus 220 is designed for infant children. For example, when a child falls asleep in a car seat, the child's head generally falls forward leaving them in an awkward position. With use of the support apparatus 220, such an awkward position may be prevented.

The resilient shaped block 230 may be formed of the same material and have the same properties as the resilient tapered block 30 as described herein with reference to FIGS. 1-4. The resilient shaped block 230, however, may have a different shape than the resilient tapered block 30.

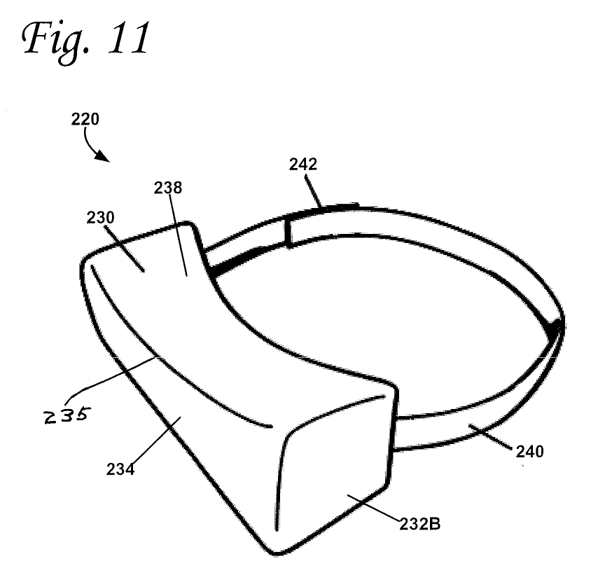

The resilient shaped block 230 may extend from a proximal surface 236 to a distal surface 234. The proximal surface 236 may be positioned adjacent the neck of the user 200 when the resilient shaped block 230 is positioned underneath the mandible of the user 200. As shown in FIGS. 11 & 14, proximal surface 236 of the resilient shaped block 230 extends along a length of the block 230 and may define a generally concave surface. In at least one embodiment, the proximal surface 236 may define a shape that conforms to the neck of the user. Further, due to the flexibility of the material of the resilient shaped block 230, the proximal surface 236 may naturally conform to the neck of the user when the support apparatus 220 is worn. Also, in at least one embodiment, the resilient shaped block 230 may be a partial ring (e.g., a banana shaped) that extends partially around the neck of the user 200.

The depth of the block 230 (e.g., between the proximal surface 236 and the distal surface 234), measured in the same manner as described with respect to FIGS. 1-4, may be less than about 2 inches or less than about 3 inches. At least in one or more embodiments, the depth is generally more than about 1 inch.

Further, the resilient shaped block 230 includes upper and lower surfaces 238, 239. In the embodiment depicted, the upper surface 238 defines a concave shape upon which the mandible of the user may rest. Further, the concave shape of the upper surface 238 may provide some support to a user who may want to roll his/her head sideways. However, the shape may also be planar or have some other curvature associated therewith. At least in one embodiment, the shaped block may include two concave surfaces (e.g. one for positioning adjacent the neck and the other to be positioned under the mandible). Further, at least in one embodiment, two or more of the surfaces may be a single piece of material covering multiple surfaces of an underlying block of material defining the two or more surfaces (e.g., in other words, an outer covering of the block may not include noticeable joints where the surfaces underlying such covering are defined). This may be the case for all embodiments of blocks or shaped blocks described herein. Yet further, in one or more embodiments, one surface may be a portion of a larger surface (e.g., the proximal surfaces may be a portion of a larger surface that includes the proximal surface, and the upper and lower surfaces together).

Further, like described with reference to FIGS. 1-4, at least in one embodiment, the upper surface 238 and the distal surface 234 intersect along a mandibular junction 235 of the block 230. The block 230 maybe sized such that when the block 230 is positioned underneath the mandible of the user 200, the mandibular junction 235 is located under the mandible of the user 200 (e.g., does not extend forward of the chin of the user).

Still further, the resilient shaped block 230 may include side surfaces 232A & 232B. In at least one embodiment, the width (e.g., between the first side surface 232A and the second side surface 232B), measured in the same manner as described with respect to FIGS. 1-4, may be less than about 8 inches, less than about 6 inches, or less than about 4 inches. Further, the width may be more than about 2 inches or more than about 3 inches. In one or more embodiments the side surfaces, and proximal and distal surfaces may form a single surface with upper and lower surfaces shaping the support block.

The support apparatus 220 further includes retention apparatus 240. The retention apparatus 240 may be formed of any suitable material, for example, skin compatible material such as, e.g., cotton, wool, nylon, polymer, neoprene, etc. Further, retention apparatus 220 may be resilient, i.e., it may be capable of returning to original form or position after being bent, compressed, stretched, etc. Still further, the resilient retention apparatus 234 may be configured to position the resilient shaped block underneath the mandible of the user and to be coupled about at least a portion of the neck of the user. In at least one embodiment, the retention apparatus 240 may wrap about three quarters of the neck of the user. The retention apparatus 240 may be substantially similar to the apparatus described herein with reference to FIGS. 1-9 (e.g., band elements, neck elements, elastic bands, covered memory metal configurations that only go around part of the neck, etc.). Further, the band elements may include apparatus for adjusting the length of such band elements when the retention apparatus 240 is positioned about the neck (e.g., strap length adjusters such as back packs, luggage straps, purse straps, etc.).

The retention apparatus 240 further may include fastener apparatus 242. The fastener apparatus 242 may be substantially similar to the closure mechanisms or fastener apparatus described herein with reference to FIGS. 1-9 (e.g., hook and loop closures, closures that release upon pulling on the block 230, snaps, ties, elastic elements that wrap around part of the neck, etc.)

The complete disclosure of the patents, patent documents, and publications cited in the Background, the Summary, the Detailed Description of Exemplary Embodiments, and elsewhere herein are incorporated by reference in their entirety as if each were individually incorporated. Exemplary embodiments of the present invention are described above. Those skilled in the art will recognize that many embodiments are possible within the scope of the invention. Other variations, modifications, and combinations of the various components and methods described herein can certainly be made and still fall within the scope of the invention. Thus, the invention is limited only by the following claims and equivalents thereto.

What is claimed is:

1. A training apparatus comprising:
a resilient tapered block positionable underneath the mandible of a user, wherein the resilient tapered block is compressible under application of a force from a normal state to a compressed state, wherein the resilient tapered block returns to the normal state when the force is removed, wherein the resilient tapered block comprises:
a proximal surface, wherein the proximal surface is positioned adjacent the neck of the user when the resilient tapered block is positioned underneath the mandible of the user;
a distal surface; and
upper and lower divergent surfaces, wherein the upper and lower divergent surfaces diverge such that the distance therebetween increases when moving from the proximal surface to the distal surface, wherein the upper divergent surface is adjacent the mandible of the user when the resilient tapered block is positioned underneath the mandible of the user with the proximal surface adjacent the neck of the user, wherein the upper divergent surface and the distal surface intersect along a mandibular junction of the resilient tapered block, wherein the mandibular junction is located under the mandible of the user when the resilient tapered block is positioned underneath the mandible of the user; and
retention apparatus configured to position the resilient tapered block underneath the mandible of the user, wherein the retention apparatus is configured to be coupled about at least a portion of the neck of the user.

2. The training apparatus of claim 1, wherein a depth of the resilient tapered block is less than 3 inches.

3. The training apparatus of claim 1, wherein the width of the resilient tapered block is less than 6 inches.

4. The training apparatus of claim 1, wherein the proximal surface is parallel to the distal surface.

5. The training apparatus of claim 1, wherein the resilient tapered block is compressible to a compressed state that occupies 80% or less volume than occupied by the resilient tapered block in the normal state.

6. The training apparatus of claim 1, wherein the resilient tapered block is compressible to a compressed state that occupies 50% or less volume than occupied by the resilient tapered block in the normal state.

7. The training apparatus of claim 1, wherein the retention apparatus comprises one or more band elements coupled to the resilient tapered block that extends at least partially around the neck of the user when the user is wearing the training apparatus.

8. The training apparatus of claim 7, wherein the retention apparatus comprises hook and loop closures configured to couple the resilient tapered block around the neck of the user when the user is wearing the training apparatus.

9. The training apparatus of claim 1, wherein the retention apparatus comprises a neck guard comprising mounting apparatus for receiving a resilient tapered block; and further wherein the resilient tapered block is removably mountable on the neck guard using the mounting apparatus.

10. The training apparatus of claim 9, wherein the mounting apparatus of the retention apparatus comprises hook and loop closures configured to couple the resilient tapered block to the neck guard.

11. A training apparatus couplable to a neck guard apparatus, the training apparatus comprising:
a resilient tapered block positionable underneath the mandible of a user, wherein the resilient tapered block is compressible under application of a force from a normal state to a compressed state, wherein the resilient tapered block returns to the normal state when the force is removed, wherein the resilient tapered block comprises:
a proximal surface, wherein the proximal surface is positioned adjacent the neck of the user when the resilient tapered block is positioned underneath the mandible of the user;
a distal surface; and
upper and lower divergent surfaces, wherein the upper and lower divergent surfaces diverge such that the distance therebetween increases when moving from the proximal surface to the distal surface, wherein the upper divergent surface is adjacent the mandible of the user when the resilient tapered block is positioned underneath the mandible of the user with the proximal surface adjacent the neck of the user, wherein the upper divergent surface and the distal surface intersect along a mandibular junction of the resilient tapered block, wherein the mandibular junction is located under the mandible of the user when the resilient tapered block is positioned underneath the mandible of the user; and
fastener apparatus located on the proximal surface configured to couple the resilient tapered block to the neck guard.

12. The training apparatus of claim 11, wherein a depth of the resilient tapered block is less than 3 inches.

13. The training apparatus of claim 11, wherein the width of the resilient tapered block is less than 6 inches.

14. The training apparatus of claim 11, wherein the proximal surface is parallel to the distal surface.

15. A method of using a training apparatus, the method comprising: providing a training apparatus, wherein the training apparatus comprises:
a resilient tapered block positionable underneath the mandible of a user, wherein the resilient tapered block is compressible under application of a force from a normal state to a compressed state, wherein the resilient tapered block returns to the normal state when the force is removed, wherein the resilient tapered block comprises:
a proximal surface;
a distal surface; and
upper and lower divergent surfaces, wherein the upper and lower divergent surfaces diverge such that the distance therebetween increases when moving from the proximal surface to the distal surface, wherein the upper divergent surface intersects with the distal surface along a mandibular junction of the resilient tapered block; and
retention apparatus configured to position the resilient tapered block underneath the mandible of the user, wherein the retention apparatus is configured to be coupled about at least a portion of the neck of the user; locating the resilient tapered block underneath the mandible of the user,
wherein the proximal surface is positioned adjacent the neck of the user, wherein the upper divergent surface is adjacent the mandible of the user, and further wherein the mandibular junction is located under the mandible of the user; and
coupling the retention apparatus about at least a portion of the neck of the user.

16. The method of claim 15, wherein the depth of the resilient tapered block is less than 3 inches.

17. The method of claim 15, wherein the width of the resilient tapered block is less than 6 inches.

18. The method of claim 15, wherein the retention apparatus comprises one or more band elements coupled to the resilient tapered block, and wherein coupling the retention apparatus about at least a portion of the neck of the user comprises connecting the one or more band elements at least partially around the neck of the user.

19. The method of claim 15, wherein the retention apparatus comprises a neck guard comprising mounting apparatus for receiving a resilient tapered block; and further wherein the resilient tapered block is removably mountable on the neck guard using the mounting apparatus.

20. A support apparatus comprising:
a resilient shaped block positionable underneath the mandible of a user, wherein the resilient shaped block is compressible under application of a force from a normal state to a compressed state, wherein the resilient shaped block returns to the normal state when the force is removed, wherein the resilient shaped block comprises:
a proximal surface extending along a length of the resilient shaped block, wherein the proximal surface is positioned adjacent the neck of the user when the resilient shaped block is positioned underneath the mandible of the user;
a distal surface; and
upper and lower surfaces extending from the proximal surface to the distal surface, wherein the upper surface is concave shaped, wherein the upper surface is adjacent the mandible of the user when the resilient shaped block is positioned underneath the mandible of the user, wherein the upper surface and the distal surface intersect along a mandibular junction of the resilient shaped block, wherein at least a portion of the mandibular junction is located under the mandible of the user when the resilient shaped block is positioned underneath the mandible of the user; and
retention apparatus configured to position the resilient shaped block underneath the mandible of the user, wherein the retention apparatus is configured to be coupled about at least a portion of the neck of the user.
